(19) United States
(12) Patent Application Publication
(10) Pub. No.: US 2024/0180935 A1
(43) Pub. Date: Jun. 6, 2024

(54) SOLUTION FORMULATION OF CYCLOPHOSPHAMIDE

(71) Applicant: Navinta, LLC, Ewing, NJ (US)

(72) Inventors: Bhaveshkumar Anilkumar Patel, Bensalem, PA (US); Mahendra R. Patel, Delray Beach, FL (US)

(21) Appl. No.: 18/517,285

(22) Filed: Nov. 22, 2023

Related U.S. Application Data
(60) Provisional application No. 63/427,226, filed on Nov. 22, 2022.

(51) Int. Cl. A61K 31/675; A61K 47/10; A61K 47/20

(52) U.S. Cl. CPC A61K 31/675; A61K 47/10; A61K 47/20

(57) ABSTRACT

The present disclosure provides stable liquid formulations of cyclophosphamide. The stable liquid formulations are free from co-solvents and acidifying agents. The stable liquid formulations cyclophosphamide contains cyclophosphamide monohydrate or cyclophosphamide anhydrous, ethanol, and one or more antioxidants.

### CROSS-REFERENCE TO RELATED APPLICATION

This application claims priority to U.S. Provisional Patent Application No. 63/427,226 filed on Nov. 22, 2022, and which is incorporated by reference in its entirety herein.

### FIELD

The present disclosure is related to stable liquid formulations of cyclophosphamide for parenteral infusion and administration.

### BACKGROUND

Cyclophosphamide is chemically known as 2-[bis(2-chloroethyl) amino]-tetrahydro-2H-1,3,2-oxazaphosphorine-2-oxide monohydrate, a widely used antineoplastic drug which is chemically classified as a nitrogen mustard. Cyclophosphamide monohydrate has the chemical structure of formula (I).

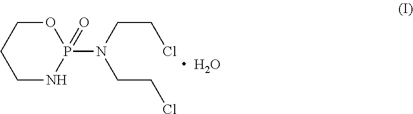

Cyclophosphamide is one example of a group of cyclic phosphoric acid ester amides which were originally disclosed and claimed in U.S. Pat. No. 3,018,302 issued Jan. 23, 1962, the entire content of which is incorporated by reference herein. It has been known for a very long time that solid cyclophosphamide is stable at room temperature as monohydrate. The first cyclophosphamide formulation was available as a lyophilized powder with about 5% water. Manufacture of this formulation required controlled absorption of moisture by lyophilized solid at the end of lyophilization cycle. Subsequently, a sterile powder form of cyclophosphamide monohydrate was developed and is now used for injection. Sterile cyclophosphamide monohydrate is now sold under various proprietary names, for example, CYTOXAN®, ENDOXAN® and NEOSAR®.

There has been a need in the art in developing a liquid formulation of cyclophosphamide that will circumvent the problems associated with both solid formulations. As far back as 1989, a liquid formulation of cyclophosphamide was disclosed in U.S. Pat. No. 4,879,286, which disclosed cyclophosphamide solutions in a mixture of 80 percent propylene glycol and about 20 percent polyethylene glycol. More recently, U.S. Pat. No. 10,993,952 described a stable, ready-to-use cyclophosphamide liquid formulation comprising cyclophosphamide dissolved in a similar solvent mixture plus ethanol and an antioxidant. In addition, U.S. Pat. No. 9,662,342 described the formulation of cyclophosphamide solution in, ethanol with ethanol soluble acid such as citric acid.

Lyophilized compositions of cyclophosphamide are also known in the art. For example. U.S. Pat. No. 4,537,883 discloses various lyophilizates of cyclophosphamide prepared by lyophilizing a solution of cyclophosphamide and one or more excipients and rehydrating the product such that it contains about 4% moisture. Kovalcik et al have also examined the stability of cyclophosphamide in lyophilized cakes containing mannitol, lactose, and sodium bicarbonate and urea, polyvinylpyrrolidone and dextran. See Kovalcik et al., “The Stability of Cyclophosphamide in Lyophilized Cakes: Part I. Mannitol, Lactose, and Sodium Bicarbonate as Excipients, (1988) PDA J Pharm Sci and Tech 42: pp. 29-37; Kovalcik et al., “Stability of cyclophosphamide in lyophilized cakes: II. Urea, polyvinylpyrrolidone, and dextran as excipients,” (1988) PDA J Parenteral Sci Tech 42(5): pp. 165-173. It is also known that cyclophosphamide hydrolyzes in water to form four major degradation products described in the USP monograph as Related Compounds (RC) A, B, C, and D. The mechanisms involving a direct hydrolysis for Cyclophoshamide and hydrolysis to RC-A and an intermediate which is further hydrolyzed to RC-C as shown in Scheme I. An alternative pathway involving internal displacement of Cl followed by hyrolysis results in a nine-membered ring compound RC-B and subsequent hydrolysis of RC-B to RC-D has been postulated by Friedman (J. Amer. Chem. Soc. 1965, 87, 4978-9) and subsequently refined by Gilard et al (J. Med. Chem. 1994, 37, 3986-93), as summarized in scheme II below. Presence of water has been understood as detrimental to product stability.

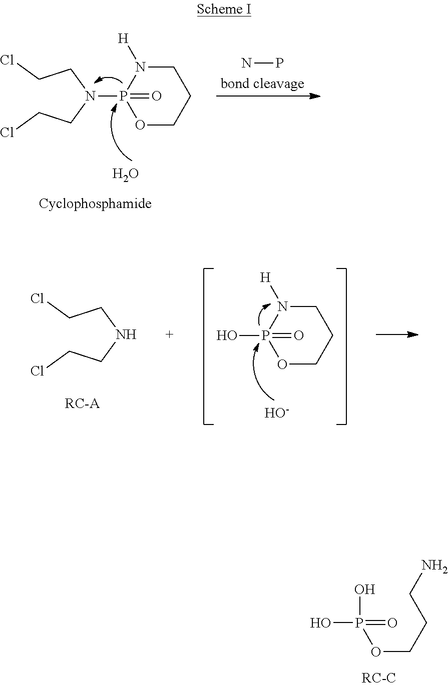

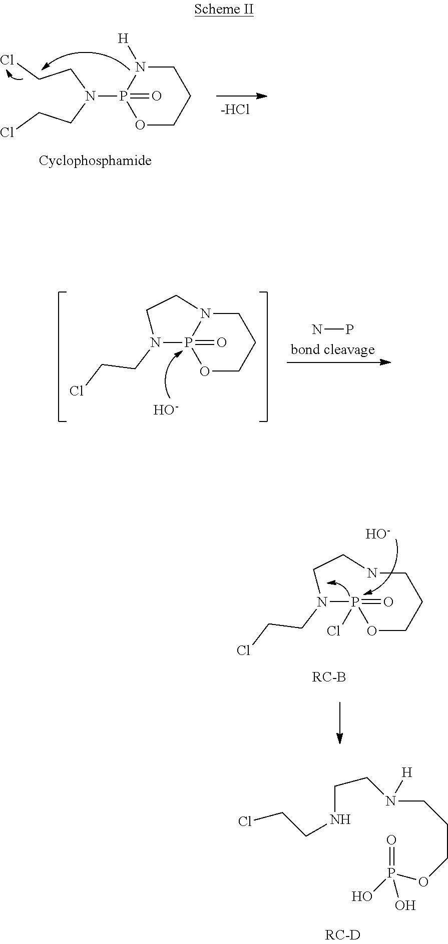

In addition, U.S. Pat. No. 4,952,575 teaches an absence of water in a liquid formulation by using 100% Ethanol as the only ingredient.

Currently, commercially available parenteral dosage formulations of cyclophosphamide comprise sterile packaged dry powder fill of cyclophosphamide monohydrate. The sterile powder is dissolved in water or normal saline prior to administration. It is intended that the solution itself be administered promptly after being prepared but it is suitable for use in several hours after preparation. During processing and/or storage of the currently available dry powder formulation, the product can acquire a glassy and/or sticky nature resulting in an undesirable material with prolonged dissolution times and decreased potency. This deterioration is more pronounced as storage time is extended or if the upper limit of the storage temperature range is exceeded.

A common practice used with constitution of sterile solids by a suitable aqueous vehicle comprises warming the solution in the container to expedite the dissolution process, especially when the solids dissolve slowly. However, it has been shown that warming vials of cyclophosphamide to facilitate dissolution after adding an aqueous vehicle could decrease the potency of the final injectable product. See Brooke et al., “Effect of briefly heating cyclophosphamide solutions, (1975) Am J Hosp Pharm, 32: pp. 44-45.

In summary, these stability limitations and dissolution difficulties can often result in clinical usage of sub-potent cyclophosphamide solutions. Sterile powder formulations require reconstitution with a diluent liquid which decreases the ease of administration. Moreover, the reconstituted and diluted solutions can be stored only for a short period (few hours) without compromising the quality of the product. Whereas sterile liquid formulations are known to require multiple cosolvents and/or antioxidants in addition to an ethanol soluble acid. However, these formulations show noticeable degradation with respect to the known and unknown impurities while monitoring the stability at 40° C./75% RH for 7 days, and at the 2-8° C.

### SUMMARY

Hence there is a need to develop liquid formulations of cyclophosphamide overcoming the disadvantages of powder products. Secondly, it is desired to have simple formulation which is easy to manufacture and has better stability as compared to other reported liquid formulations of cyclophosphamide. The present disclosure addresses these needs.

The present disclosure provides stable liquid formulations of cyclophosphamide for parenteral infusion and administration.

More specifically, an object of the present disclosure is to provide liquid cyclophosphamide solution for infusion with improved stability characteristics.

Yet another aspect of the present disclosure is to provide stable ready to use liquid parenteral formulation of cyclophosphamide without cosolvent(s) and ethanol soluble acidifying agent. Cyclophosphamide containing compositions such as pharmaceutically acceptable cyclophosphamide containing solutions are provided having extended stability. Compositions include a) cyclophosphamide monohydrate or cyclophosphamide anhydrous; b) ethanol; and c) antioxidant(s).

Also provided is a solvent system which contains only ethanol and excipients such as monothioglycerol, butylated hydroxytoluene or a combination thereof.

In embodiments, a stable cyclophosphamide solution for parenteral administration comprises cyclophosphamide in the form of pharmaceutically acceptable hydrate and/or anhydrous forms in ethanol at about 2% w/w to 50% w/w and water at a level between 2.0% and 5.0%.

In embodiments, the ethanol is anhydrous ethanol.

In embodiments, the solution further comprises an antioxidant.

In embodiments, the antioxidant is selected from butylated hydroxyanisole, butylated hydroxytoluene, monothioglycerol, and a combination of two or more thereof.

In embodiments, the solution comprises from 5% w/w to 40% w/w cyclophosphamide monohydrate; and from 60% w/w to 95% w/w dehydrated alcohol.

In embodiments, the solution comprises from 2% w/w to 40% w/w cyclophosphamide monohydrate; from 20% w/w to 99% w/w dehydrated alcohol; and from 0.001% w/w to 1% w/w butylated hydroxytoluene.

In embodiments, the solution comprises from 2% w/w to 40% w/w cyclophosphamide monohydrate; from 20% w/w to 99% w/w dehydrated alcohol; and from 0.01% w/w to 5% w/w monothioglycerol.

In embodiments, the solution comprises from 2% w/w to 40% w/w cyclophosphamide monohydrate; from 20% w/w to 99% w/w dehydrated alcohol; from 0.001% w/w to 1% w/w butylated hydroxytoluene; and from 0.01% w/w to 5% w/w monothioglycerol.

In embodiments, the solution comprises from 2% w/w to 50% w/w cyclophosphamide monohydrate; from 20% w/w to 99% w/w dehydrated alcohol; and from 2% w/w to 5% w/w water.

In embodiments, the solution comprises from 2% w/w to 50% w/w cyclophosphamide monohydrate; from 20% w/w to 99% w/w dehydrated alcohol; from 2% w/w to 5% w/w water; and from 0.01% w/w to 5% w/w monothioglycerol.

In embodiments, the solution comprises about 22.2% w/w cyclophosphamide monohydrate; about 74.81% w/w dehydrated alcohol; and about 3% w/w water.

In embodiments, the solution further comprises a pharmaceutically acceptable carrier.

In embodiments, the pharmaceutically acceptable carrier is selected from the group consisting of sugar; starch; cellulose and its derivatives; powdered tragacanth; malt; gelatin; talc; excipients; oils; glycols; esters; agar; buffering agents; alginic acid; pyrogen-free water; isotonic saline; Ringer's solution; ethyl alcohol; phosphate buffer solutions; lubricants; coloring agents; releasing agents; coating agents; sweetening agents; flavoring agents; perfuming agents; preservatives; antioxidants; and a combination of two or more thereof.

In embodiments, the solution further comprises a stabilizer.

In embodiments, a stable cyclophosphamide solution for parenteral administration comprises cyclophosphamide in the form of pharmaceutically acceptable hydrate and/or anhydrous forms in ethanol at about 5% w/w to 50% w/w, water at a level between 2.0% and 5.0% and one or more antioxidants at a level of monothioglycerol at about 0.01% to 5% w/w and butylated hydroxytoluene at about 0.001% to 1% w/w.

These and other embodiments and features of the disclosure will become more apparent through reference to the following description, the accompanying figures, and the claims. Furthermore, it is to be understood that the features of the various embodiments described herein are not mutually exclusive and can exist in various combinations and permutations.

### DETAILED DESCRIPTION

The present disclosure provides novel stable formulations for cyclophosphamide for parenteral administration. The term “cyclophosphamide” as used herein refers to pharmaceutically acceptable active ingredient and widely used anticancer agent in the form of pharmaceutically acceptable salts, solvates, hydrates and anhydrous forms thereof, such as cyclophosphamide monohydrate or anhydrous cyclophosphamide. Some broad aspects of the disclosure include cyclophosphamide containing compositions which comprise cyclophosphamide, ethanol, and optionally antioxidant(s). In some embodiments, the cyclophosphamide containing compositions are in the form of a substantially non-aqueous, ethanolic solution which is ready for dilution and administration to a patient in need thereof.

Cyclophosphamide is known to be susceptible to hydrolysis. Therefore, it is supplied as a lyophilized formulation as well as in a form of sterile powder and non-aqueous liquid formulation to reduce the formation of impurities and to improve the stability of the final formulation.

The inventors have discovered a stable, ready to use, liquid parenteral formulation of cyclophosphamide which is free from ethanol-soluble acid as well as any co-solvents and which has impurities controlled within the acceptable limits. Additionally, inventors find that added water in the formulation is not as detrimental to stability as previously believed in the pertinent field.

The solvent included in the compositions of the present disclosure can be any type of ethanol, such as an ethanol which meets the requirements of the U.S. or European Pharmacopoeia. In certain aspects of the disclosure the ethanol is anhydrous. The quantity of solvent ranges from about 40%-99% by weight of the composition.

Optional, antioxidant(s) included in the compositions of the present disclosure may be selected from butylated hydroxyanisole, butylated hydroxytoluene, monothioglycerol and the like. The concentration of the antioxidant used is less than 5%, such as less than 3% by weight of the composition.

In one embodiment, the liquid formulations of cyclophosphamide comprise cyclophosphamide monohydrate and one or more antioxidants. In some embodiments, the one or more antioxidants may be selected from butylated hydroxytoluene, monothioglycerol, butylated hydroxyanisole, and the like. Further, a stable solution formulation of cyclophosphamide comprising cyclophosphamide monohydrate or cyclophosphamide anhydrous and at least one antioxidant selected from butylated hydroxy anisole, butylated hydroxytoluene and monothioglycerol were tested for indicative stability at 40° C./75% RH for 7 days.

In some aspects, the liquid formulations of cyclophosphamide comprises cyclophosphamide monohydrate or cyclophosphamide anhydrous, and one or more antioxidants. In some embodiments, the antioxidants may be selected from the group of antioxidants, butylated hydroxy anisole, and butylated hydroxytoluene.

Further, solution formulations of cyclophosphamide comprising any one the antioxidants selected from butylated hydroxyanisole, butylated hydroxytoluene, and monothioglycerol were tested for stability at 40° C./75% RH for 7 days.

For the avoidance of doubt, for purposes of the following stable solution formulations of cyclophosphamide, the total % w/w for all components of the formulation, including the explicitly listed components, will be 100% w/w.

One embodiment of stable solution formulation of cyclophosphamide comprises:

TABLE 1
Composition A

| Sr. no | Material | % w/w |
|---|---|---|
| 1 | Cyclophosphamide monohydrate | 5-40% |
| 2 | Dehydrated alcohol | 60-95% |

In another embodiment, the stable solution formulation of cyclophosphamide comprises:

TABLE 2
Composition B

| Sr. no | Material | % w/w |
|---|---|---|
| 1 | Cyclophosphamide monohydrate | 2-40% |
| 2 | Dehydrated alcohol | 20-99% |
| 3 | Butylated hydroxytoluene | 0.001-1% |

In another embodiment, the stable solution formulation of cyclophosphamide comprises:

TABLE 3
Composition C

| Sr. no | Material | % w/w |
|---|---|---|
| 1 | Cyclophosphamide monohydrate | 2-40% |
| 2 | Dehydrated alcohol | 20-99% |
| 3 | Monothioglycerol | 0.01-5% |

In yet another embodiment, the stable solution formulation of cyclophosphamide comprises:

TABLE 4
Composition D

| Sr. no | Material | % w/w |
|---|---|---|
| 1 | Cyclophosphamide monohydrate | 2-40% |
| 2 | Dehydrated alcohol | 20-99% |
| 3 | Butylated hydroxytoluene | 0.001-1% |
| 4 | Monothioglycerol | 0.01-5% |

In yet another embodiment, the stable solution formulation of cyclophosphamide comprises:

TABLE 5
Composition E

| Sr. no | Material | % w/w |
|---|---|---|
| 1 | Cyclophosphamide monohydrate | 2-40% |
| 2 | Dehydrated alcohol | 20-99% |
| 3 | Water for injection | 2-5% |

In yet another embodiment, the stable solution formulation of cyclophosphamide comprises:

TABLE 6
Composition F

| Sr. no | Material | % w/w |
|---|---|---|
| 1 | Cyclophosphamide monohydrate | 2-40% |
| 2 | Dehydrated alcohol | 20-99% |
| 3 | Water for injection | 2-5% |
| 4 | Monothioglycerol | 0.01-5% |

In still another embodiment, the stable solution formulation of cyclophosphamide comprises:

TABLE 7
Composition G

| Sr. no | Material | % w/w |
|---|---|---|
| 1 | Cyclophosphamide monohydrate | 22.2% |
| 2 | Dehydrated alcohol | 74.81% |
| 3 | Water for injection | 3% |

For comparison, the prior art liquid formulations of Cyclophosphamide monohydrate dissolved in a solvent system comprising alcoholic solvents and antioxidant (s) were tested for indicative stability at 40° C./75% RH for 7 days and impurity profiles were compared with the liquid formulations of the present disclosure.

The inventors also compared the prior art liquid formulation of cyclophosphamide monohydrate, in ethanol, and citric acid as acidifying agent in indicative stability at 40° C./75% RH for 7 days, and the impurity profile was compared with the liquid formulations of the present disclosure. The composition details and stability data at 40° C./75% RH of the liquid formulation as per U.S. Pat. Nos. 4,879,286 and 10,993,952 are summarized in below table 8 and 9. The formulations are summarized in the below table along with their stability at 40° C. for at least 1 week as reported in those patents:

TABLE 8
Compositions reported in U.S. Pat. No. 4,879,286

| Example no: | 10 | 11 |
|---|---|---|
| Propylene glycol | 80% | 80% |
| Polyethylene glycol | 20% | 20% |
| Glycerol | — | — |
| Water for injection | — | — |
| Cyclophosphamide (mg/mL) | 20 | 100 |

Assay result after 1 week at 40° C.

| | | |
|---|---|---|
| Initial | 100 | 100 |
| 1 week | 86.5 | — |
| 2 weeks | 71.5 | 78.7 |

TABLE 9
Compositions reported in U.S. Pat. No. 10,993,952:

| Composition Code | Example 2 | Example 4 | Example 5 |
|---|---|---|---|
| Material | g/vial | g/vial | g/vial |
| Cyclophosphamide | 1.0 | 1.0 | 1.0 |
| Dehydrated alcohol | 6.23 | 6.95 | 3.15 |
| Polyethylene Glycol-400 | 0.733 | 0.310 | 0.150 |
| Propylene glycol | 0.367 | — | — |
| Monothioglycerol | 0.69 | 0.69 | 0.69 |

Analytical result after 1 week at 40° C.

| | | | |
|---|---|---|---|
| Assay | 101.9 | 98.9 | 99.7 |
| Total Impurity | 1.87% | 2.01% | 2.33% |

Cyclophosphamide formulations prepared according to the disclosure were tested for stability under accelerated conditions for a period of 1 week at 40° C./75% RH.

### Definitions

Various quantities, such as amounts, sizes, dimensions, proportions, and the like, are presented in a range format throughout this disclosure. It should be understood that the description of a quantity in range format is merely for convenience and brevity and should not be construed as an inflexible limitation on the scope of any embodiment. Accordingly, the description of a range should be considered to have specifically disclosed all the possible subranges as well as all individual numerical values within that range unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 4.62, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is to describe particular embodiments only and is not intended to be limiting of any embodiment. As used herein, the singular forms “a,” “an”, and “the” are intended to include the plural forms as well, unless the context clearly indicates otherwise. It will be further understood that the terms “includes”, “comprises”, “including” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Unless expressly stated or obvious from context, as used herein, the term “about” in reference to a number or range of numbers is understood to mean the stated number and numbers+/−10% thereof, or 10% below the lower listed limit and 10% above the higher listed limit for the values listed for a range.

In any of the embodiments disclosed herein, the terms “treating” or “to treat” includes restraining, slowing, stopping, or reversing the progression or severity of an existing symptom or disorder.

In any of the embodiments disclosed herein, the term “patient” refers to a human.

“Effective or Therapeutic Amount”

Effective or therapeutic amounts of any of the drugs or pharmaceutical compositions of this disclosure include any amount sufficient to inhibit (e.g., slow or stop) sufficient to inhibit a tumor or growth of cancerous cells. The amount of the active ingredient that may be combined with the optional carrier materials to produce a single dosage form may vary depending upon the host treated and the particular mode of administration. The specific dose level for any particular patient may depend upon a variety of factors, including the activity of the specific compound employed, the age, body weight, general health, sex, diet, time of administration, route of administration, rate of excretion, drug combination, and the severity of the particular disorder or disease undergoing therapy. A therapeutically effective amount for a given situation can be readily determined by routine experimentation and is within the skill and judgment of the ordinary clinician.

Pharmaceutically Acceptable Carriers and Other Excipients

As used herein, the term “pharmaceutically acceptable carrier” means a non-toxic, inert solid, semi-solid or liquid filler, diluent, encapsulating material or formulation auxiliary of any type. In the treatment methods contemplated by the present disclosure, the disclosed agents may be used alone or in compositions together with a pharmaceutically acceptable carrier or excipient, such as saline. For example, an oral dosage form composition may comprise one or more of the disclosed agents in addition to a pharmaceutically acceptable carrier.

Some examples of materials which can serve as pharmaceutically acceptable carriers are sugars, such as lactose, glucose and sucrose; starches, such as corn starch and potato starch; cellulose and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil; safflower oil; sesame oil; olive oil; corn oil and soybean oil; glycols, such as propylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol, and phosphate buffer solutions, as well as other non-toxic compatible lubricants, such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Other suitable pharmaceutically acceptable excipients are described in “Remington's Pharmaceutical Sciences,” Mack Pub. Co., New Jersey, 1991, the contents of which are expressly incorporated herein by reference.

“Purified”

The term “purified” as used herein refers to material that has been isolated under conditions that reduce or eliminate the presence of unrelated materials, i.e. contaminants, including native materials from which the material is obtained. As used herein, the term “substantially free” is used operationally, in the context of analytic testing of the material. In some embodiments, purified material substantially free of contaminants is at least 95% pure, such as at least 97% pure or even at least 99% pure. Purity can be evaluated for example by chromatography or any other methods known in the art. In particular embodiments, purified means that the level of contaminants is below a level acceptable to regulatory authorities for safe administration to a human or a non-human animal.

“Pharmaceutically Acceptable”

The term “pharmaceutically acceptable,” when used in connection with the pharmaceutical compositions of the application, refers to molecular entities and compositions that are physiologically tolerable, and do not typically produce untoward reactions when administered to a human. In some embodiments, as used herein, the term “pharmaceutically acceptable” may mean approved by a regulatory agency of the Federal or state government or listed in the U.S. Pharmacopeia or other generally recognized pharmacopeia for use in animals, and more particularly in humans. The term “carrier” refers to a diluent, excipient, dispersing agent or vehicle with which the composition is administered. Such pharmaceutical carriers can be sterile liquids, such as water and oils. For example water, aqueous solutions, saline solutions or aqueous glycerol solutions can be employed as carriers, particularly for injectable solutions. Suitable pharmaceutical carriers are described in, for example, “Remington's Pharmaceutical Sciences” by Philip P. Gerbino, 22nd Edition). Aqueous solutions are those in which water is the solvent. The term “pharmaceutical composition” as used in accordance with the present application relates to compositions that can be formulated in any conventional manner using one or more pharmaceutically acceptable carriers or excipients.

“Dosage”

The term “dosage” is intended to encompass a formulation expressed in terms of μg/kg/day, μg/kg/hr., mg/kg/day or mg/kg/hr. The dosage is the amount of an ingredient administered in accordance with a particular dosage regimen. A “dose” is an amount of an agent administered to a mammal in a unit volume or mass, e.g., an absolute unit dose expressed in mg or μg of the agent. The dose depends on the concentration of the agent in the formulation, e.g. in moles per liter (M), mass per volume (m/v) or mass per mass (m/m). The two terms are closely related, as a particular dosage results from the regimen of administration of a dose or doses of the formulation. The particular meaning in any case will be apparent from context.

Buffers

Buffer systems for use in the present embodiments may include citrate, acetate, bicarbonate and phosphate buffers. Suitable buffers are generally buffers that stabilize the pH of the contemplated liquid formulations in an acidic pH range and will therefore include glycine buffers, citrate buffers, citrate/phosphate buffers, acetate buffers, etc. However, the inventors have further discovered that where the phenylephrine is provided as the phenylephrine HCl salt, a buffer can advantageously be omitted and the pH can be adjusted with suitable acid and/or base as is well known in the art. Most typically the pH of the formulation will be less than 5.0 and more typically less than 4.5, and most typically less than 4.3, but higher than 3.0, more typically about 3.5. For example, suitable buffers will have a pH in the range of between 3.7 and 4.3, or between 3.7 and 4.0, or between 3.8 and 4.1, or between 3.9 and 4.2, or between 4.0 and 4.2. Notably, such pH range provided remarkable stability for low concentrations of phenylephrine, especially when in combination with a chelator and a salt. While not limiting to the inventive subject matter, the buffer strength is typically relatively low, for example, equal or less than 300 mM, and more typically equal or less than 200 mM, and most typically between 175 mM and 20 mM (e.g., 154 mM, i.e. 0.9% NaCl). Furthermore, phenylephrine aqueous solutions are optically stable on the pH range of 3.0 to 6.0.

Salts

With respect to suitable salts it is contemplated that the salt is a pharmaceutically acceptable salt that can be used to increase tonicity as a tonicity adjusting agent. Therefore, pharmaceutically acceptable salts are contemplated, and especially NaCl, at a concentration of at least 0.6 wt %, or at least 0.7 wt %, or at least 0.8 wt %, or at least 0.9 wt %. For example, suitable salt concentrations are between 0.6 wt % and 1.2 wt %. Depending on the particular salt concentration, additional tonicity adjusting agents may be added and suitable tonicity adjusting agents include sodium chloride, potassium chloride, sodium phosphate, potassium phosphate, glycerol, thioglycerol, mannitol, lactose, and dextrose. The amount of tonicity adjusting agent used can be adjusted to obtain osmolality of the formulations in the range of 260 to 340 mOsm/kg. An osmometer can be used to check and adjust the amount of tonicity adjusting agent to be added to obtain the desired osmolality.

Stabilizers

A composition, formulation, or dosage form herein may further comprise one or more of the disclosed agents and one or more stabilizers. As used herein, a stabilizer is a substance that extends the time before which one or more of the disclosed agents in a composition is converted to a salt in the environment in which the formulation or dosage form is administered, in comparison to the conversion in its absence. Non-limiting examples of stabilizers include phosphatidyl choline, phosphatidyl inositol, phosphatidyl ethanolamine, or other phospholipids. A composition, formulation, or dosage form further comprising one or more stabilizers may be administered in any one of the methods herein. A stabilizer may be present in an amount of about 50 mg to about 1000 mg in a composition, formulation, or dosage form herein. In some embodiments, the stabilizer may be present in an amount ranging from about 50 mg to about 500 mg or about 50 mg to about 100 mg.

Further reference is made to the following experimental examples.

### EXAMPLES

The following examples are provided for the purpose of illustrating various embodiments of the invention and are not meant to limit the present disclosure in any fashion. The present examples, along with the methods described herein are presently representative of certain embodiments, are provided only as examples, and are not intended as limitations on the scope of the invention. Changes therein and other uses which are encompassed within the spirit of the disclosure as defined by the scope of the claims will occur to those skilled in the art.

HPLC Method for Detecting Impurities in Cyclophosphamide Solution:

| | |
|---|---|
| RC-A | Bis(2-chloroethyl)amine hydrochloride |
| RC-B | 3-(2-Chloroethyl)-2-oxo-2-hydroxy-1,3,6,2- |
| | oxadiazaphosphonane |
| RC-C | 3-Aminopropyl dihydrogen phosphate |
| RC-D | 3-[2-(2-Chloroethylamino)ethylamino]propyl |
| | dihydrogen phosphate |

Analytical Method:

Method 1: HPLC Method for Cyclophosphamide Related Compounds A, B and D

To analyze degradation products of cyclophosphamide that can be present in a formulation of cyclophosphamide, gradient HPLC was performed to separate cyclophosphamide from impurities and formulation components. The concentration of impurities in the sample were determined using the external standard method, using the cyclophosphamide reference standard peak area and a relative response factor for each impurity.

TABLE 10
Experiment detailing for Cyclophosphamide
related compound A, B and D.

| Equipment | HPLC with UV detector | |
|---|---|---|
| Column | X-Bridge C18 (4.6 × 250) mm, 5 μm, (waters) | |
| Mobile Phase A | Potassium Dihydrogen Phosphate buffer, pH 8.0 | |
| Mobile Phase B | Acetonitrile: Mobile phase A (80:20) | |
| Wavelength | 195 | nm |
| Flow Rate | 0.5 | mL/min |
| Column Temperature | 25° | C. |
| Sample Temperature | 4° | C. |
| Injection Volume | 100 | μL |

| | |
|---|---|
| Diluent | Water |

Gradient Program:

| Time (min) | % Mobile phase (A) | % Mobile phase (B) |
|---|---|---|
| 0 | 100 | 0 |
| 12 | 100 | 0 |
| 30 | 70 | 30 |
| 65 | 20 | 80 |
| 75 | 20 | 80 |
| 80 | 100 | 0 |
| 90 | 100 | 0 |

TABLE 11
Details of the Retention time, Relative Retention time
(RRT) and Relative Response Factor (RRF) values

| Name | About RT (minutes) | RRT | RRF |
|---|---|---|---|
| RC-B | 7.52 | 0.17 | 1.04 |
| RC-D | 11.33 | 0.26 | 0.40 |
| RC-A | 41.55 | 0.95 | 1.01 |
| Cyclophosphamide | 43.60 | 1.00 | 1.00 |

The impurities levels were calculated by applying the response factor to the observed peak area for each impurity.

Method 2: HPLC Method for Cyclophosphamide Related Compound C

To analyze cyclophosphamide related compound C present in a formulation, gradient HPLC after derivatization with 9-Fluorenyl methyl chloroformate (FMOC-Cl) was performed. The concentration of related compound C in the sample was determined using the external standard method.

TABLE 12
Experiment detailing for related compound C.

| Equipment | HPLC with UV detector | |
|---|---|---|
| Column | Inertsil ODS-3 (4.6 × 150) mm, Particle size: 5 μm, (GL Sciences) | |
| Mobile Phase A | Potassium Dihydrogen Phosphate buffer, pH 3.0 | |
| Mobile Phase B | Acetonitrile: Methanol (80:20) | |
| Wavelength | 265 | nm |
| Flow Rate | 1.0 | mL/min |
| Column Temperature | 35° | C. |
| Sample Temperature | 5° | C. |
| Injection Volume | 20 | μL |

| | |
|---|---|
| Diluent | Borate buffer at pH 10 |

Gradient Program:

| Time (min) | % Mobile phase (A) | % Mobile phase (B) |
|---|---|---|
| 0 | 60 | 40 |
| 10 | 60 | 40 |
| 12 | 30 | 70 |
| 28 | 30 | 70 |
| 30 | 60 | 40 |
| 40 | 60 | 40 |

Derivatization solution was prepared by transferring 500.0 mg of 9-Fluorenyl methyl chloroformate (FMOC-Cl) into a 100 mL volumetric flask. Added about 60 mL of acetonitrile and mixed on vortex mixer to dissolve the solids. Diluted to volume with Acetonitrile and mixed well. The working standard solution contained about 1.2 μg/mL of cyclophosphamide related compound C. The working test sample solution contained about 2000 μg/mL of cyclophosphamide. Transferred 0.25 mL of test sample solution into a 25 mL volumetric flask. Added 4.5 mL of diluent, 7.5 mL of methanol, 5.0 mL of the derivatization solution and vortex for 1 minute. Diluted to volume with methanol and mix well.

TABLE 13
Retention time of Cyclophosphamide related compound C

| Name | About RT (minutes) |
|---|---|
| RC-C | 7.1 |

Total Impurities:

Total impurities=Impurity A+Impurity B+Impurity D+Impurity C+sum of all unknown impurities in method 1

### Example 1

Compounding Process for the Formulations of Our Disclosure: Composition Number A

In a suitable vessel 200 mL of Dehydrated alcohol was collected and cooled to 5° C. To another vessel, 150 mL of cooled dehydrated alcohol (5° C.) was transferred and 42.76 g of cyclophosphamide monohydrate (equivalent to 40 g of cyclophosphamide) was added. The mixture was stirred till the solids were completely dissolved and volume of the liquid was made up to 200 mL with cold ethanol. Bulk solution temperature was maintained at 5° C. throughout the compounding process. The solution was filtered through 0.2 μm filter, filled the solution in glass vials, followed by stoppering and sealing of vials.

Other compositions described in the table below were prepared by analogous methods.

TABLE 14
Compositions of cyclophosphamide solution (mg/ml)

| Composition | A | B | C | D | E | F | G | 8^{#} | 9^{#} | 10^{#} |
|---|---|---|---|---|---|---|---|---|---|---|
| Cyclophosphamide | 213.8 | 213.8 | 213.8 | 213.8 | 213.8 | 213.8 | 213.8 | 213.8 | 213.8 | 534.5 |
| monohydrate (mg) | | | | | | | | | | |
| (equivalent to | | | | | | | | | | |
| Cyclophosphamide | | | | | | | | | | |
| 200 mg or 500 mg | | | | | | | | | | |
| as applicable) | | | | | | | | | | |
| Dehydrated | QS | QS | QS | QS | QS | QS | QS | QS | QS | QS |
| alcohol | | | | | | | | | | |
| Butylated | — | — | 0.10 | 0.02 | — | 0.02 | | — | — | — |
| hydroxytoluene | | | | | | | | | | |
| (mg) | | | | | | | | | | |
| Mono-thioglycerol | — | 0.14 | — | 0.14 | — | — | | 0.14 | — | — |
| (mg) | | | | | | | | | | |
| Polyethylene | — | — | — | — | — | — | | 34 | — | — |
| Glycol-400 (mg) | | | | | | | | | | |
| Propylene Glycol | — | — | — | — | — | — | | 34 | — | — |
| (mg) | | | | | | | | | | |
| Citric acid, | — | — | — | — | — | — | | — | 4 | — |
| Anhydrous (mg) | | | | | | | | | | |
| Water (mg) | — | — | — | — | 30 | 30 | 10 | — | — | — |

*QS—Quantity sufficient.
^{#}Prior art formulations

Stability data of the current novel formulations of Cyclophosphamide solutions and formulation from prior art are provided here for comparison.

TABLE 15
Stability data

| | Stability condition: 2° C.-8° C. | | | | |
|---|---|---|---|---|---|
| | Time point | | | | |
| Composition | 1 M | 2 M | 3 M | 6 M | 9 M |
| Number ↓ | Total Impurities (% w/w) | | | | |
| A | 0.17% | 0.27% | 0.20% | NP | NP |
| B | 0.25% | 0.30 | 0.37% | NP | NP |
| C | 0.24% | 0.33% | 0.43% | NP | NP |
| F | 0.27% | 0.41% | 0.40% | NP | NP |
| G | NP | 0.16% | 0.22% | 0.65% | 0.99% |
| 8^{#} | NP | 0.41% | NP | NP | NP |
| 9^{#} | NP | 0.45% | NP | NP | NP |
| 10^{#} | 0.34% | NP | NP | NP | NP |

*NP: Not performed

#Composition 8: from U.S. Pat. No. 10,993,952, Composition 9: from U.S. Pat. No. 9,662,342. #Composition 10: derived from product leaflet of Dr. Reddy's Laboratories Inc. Formulations with numerical numbers are from earlier references whereas formulations with letters are from current disclosure.

TABLE 16
Stability data of current disclosure v/s commercial products:

| | Total impurities (% w/w) | | |
|---|---|---|---|
| Composition | Initial | 1 Week 40° C. | % Increase |
| Athenex Pharmaceutical | 1.14 | 3.42 | 2.28 |
| Division, LLC. | | | |
| (Lot # 20082) EXP date: | | | |
| 2022 November | | | |
| AuroMedics Pharma LLC | 0.95 | 2.89 | 1.94 |
| (Lot #CCY210001-A) | | | |
| EXP date: 2023 April | | | |
| Composition A | 0.13 | 2.15 | 2.02 |
| Composition B | 0.04 | 2.03 | 1.99 |
| Composition C | 0.19 | 2.09 | 1.90 |
| Composition D | 0.15 | 2.44 | 2.29 |
| Composition E | 0.16 | 2.30 | 2.14 |

TABLE 17
Impurity data of commercial products before expiry:

| | Total impurities (% w/w) |
|---|---|
| Composition | 2° C.-8° C. storage |
| INGENUS/Athenex Pharmaceutical | 1.46 |
| Division, LLC. | |
| (Lot# 21093) Exp: October 2023 | |
| AuroMedics Pharma LLC | 2.75 |
| (Lot #CCY210001-A) | |
| EXP date: 2023 April | |
| AuroMedics Pharma LLC | 2.85 |
| (Lot #CCY210004-A) | |
| EXP date: 2023 April | |

As will be appreciated from the descriptions herein, a wide variety of aspects and embodiments are contemplated by the present disclosure, examples of which include, without limitation, the aspects and embodiments listed below:

The current disclosure provides stable liquid formulations of cyclophosphamide for parenteral infusion and administration.

More specifically, the current disclosure provides:
Liquid cyclophosphamide solutions for infusion containing with improved stability characteristics;
Stable ready to use liquid parenteral formulation of cyclophosphamide without cosolvent(s) and ethanol soluble acidifying agent;
Cyclophosphamide containing compositions such as pharmaceutically acceptable cyclophosphamide containing solutions having extended stability wherein the compositions can include anhydrous cyclophosphamide or cyclophosphamide monohydrate in a solution dosage along with ethanol and one or more antioxidants;
A solvent system which contain only ethanol and excipients such as monothioglycerol, butylated hydroxytoluene or a combination thereof.

In addition to the aspects and embodiments described and provided elsewhere in the present disclosure, the following non-limiting list of embodiments are also contemplated.
1. A stable cyclophosphamide solution for parenteral administration comprising cyclophosphamide in the form of pharmaceutically acceptable hydrate and/or anhydrous forms in ethanol at about 2% w/w to 50% w/w of cyclophosphamide and water at a level between 2.0% and 5.0%.
2. The stable cyclophosphamide solution of clause 1, wherein the ethanol is anhydrous ethanol.
3. The stable cyclophosphamide solution of clause 1, further comprising an antioxidant.
4. The stable cyclophosphamide solution of clause 3, wherein the antioxidant is selected from butylated hydroxyanisole, butylated hydroxytoluene, monothioglycerol, and a combination of two or more thereof.
5. The stable cyclophosphamide solution of clause 1, comprising: from 5% w/w to 50% w/w cyclophosphamide monohydrate; and from 60% w/w to 95% w/w dehydrated alcohol.
6. The stable cyclophosphamide solution of clause 3, comprising: from 2% w/w to 40% w/w cyclophosphamide monohydrate; from 20% w/w to 99% w/w dehydrated alcohol; and from 0.001% w/w to 1% w/w butylated hydroxytoluene.
7. The stable cyclophosphamide solution of clause 3, comprising: from 2% w/w to 40% w/w cyclophosphamide monohydrate; from 20% w/w to 99% w/w dehydrated alcohol; and from 0.01% w/w to 5% w/w monothioglycerol.
8. The stable cyclophosphamide solution of clause 3, comprising: from 2% w/w to 40% w/w cyclophosphamide monohydrate; from 20% w/w to 99% w/w dehydrated alcohol; from 0.001% w/w to 1% w/w butylated hydroxytoluene; and from 0.01% w/w to 5% w/w monothioglycerol.
9. The stable cyclophosphamide solution of clause 1, comprising: from 2% w/w to 50% w/w cyclophosphamide monohydrate; from 20% w/w to 99% w/w dehydrated alcohol; and from 2% w/w to 5% w/w water.
10. The stable cyclophosphamide solution of clause 3, comprising: from 2% w/w to 50% w/w cyclophosphamide monohydrate; from 20% w/w to 99% w/w dehydrated alcohol; from 2% w/w to 5% w/w water; and from 0.01% w/w to 5% w/w monothioglycerol.
11. The stable cyclophosphamide solution of clause 1, comprising: about 22.2% w/w cyclophosphamide monohydrate; about 74.81% w/w dehydrated alcohol; and about 3% w/w water.
12. The stable cyclophosphamide solution of clause 1, further comprising a pharmaceutically acceptable carrier.
13. The stable cyclophosphamide solution of clause 12, wherein the pharmaceutically acceptable carrier is selected from the group consisting of sugar; starch; cellulose and its derivatives; powdered tragacanth; malt; gelatin; talc; excipients; oils; glycols; esters; agar; buffering agents; alginic acid; pyrogen-free water; isotonic saline; Ringer's solution; ethyl alcohol; phosphate buffer solutions; lubricants; coloring agents; releasing agents; coating agents; sweetening agents; flavoring agents; perfuming agents; preservatives; antioxidants; and a combination of two or more thereof.
14. The stable cyclophosphamide solution of clause 1, further comprising a stabilizer.
15. A stable cyclophosphamide solution for parenteral administration comprising cyclophosphamide in the form of pharmaceutically acceptable hydrate and/or anhydrous forms in ethanol at about 5% w/w to 50% w/w, water at a level between 2.0% and 5.0% and one or more antioxidants at a level of monothioglycerol at about 0.01% to 5% w/w and butylated hydroxytoluene at about 0.001% to 1% w/w.

While embodiments of the present disclosure have been described herein, it is to be understood by those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A stable cyclophosphamide solution for parenteral administration comprising cyclophosphamide in the form of pharmaceutically acceptable hydrate and/or anhydrous forms in ethanol at about 2% w/w to 50% w/w and water at a level between 2.0% and 5.0%.

2. The stable cyclophosphamide solution of claim 1, wherein the ethanol is anhydrous ethanol.

3. The stable cyclophosphamide solution of claim 1, further comprising an antioxidant.

4. The stable cyclophosphamide solution of claim 3, wherein the antioxidant is selected from butylated hydroxyanisole, butylated hydroxytoluene, monothioglycerol, and a combination of two or more thereof.

5. The stable cyclophosphamide solution of claim 1, comprising:
from 5% w/w to 40% w/w cyclophosphamide monohydrate; and
from 60% w/w to 95% w/w dehydrated alcohol.

6. The stable cyclophosphamide solution of claim 3, comprising:
from 2% w/w to 40% w/w cyclophosphamide monohydrate;
from 20% w/w to 99% w/w dehydrated alcohol; and
from 0.001% w/w to 1% w/w butylated hydroxytoluene.

7. The stable cyclophosphamide solution of claim 3, comprising:
from 2% w/w to 40% w/w cyclophosphamide monohydrate;
from 20% w/w to 99% w/w dehydrated alcohol; and
from 0.01% w/w to 5% w/w monothioglycerol.

8. The stable cyclophosphamide solution of claim 3, comprising:
from 2% w/w to 40% w/w cyclophosphamide monohydrate;
from 20% w/w to 99% w/w dehydrated alcohol;
from 0.001% w/w to 1% w/w butylated hydroxytoluene; and
from 0.01% w/w to 5% w/w monothioglycerol.

9. The stable cyclophosphamide solution of claim 1, comprising:
from 2% w/w to 50% w/w cyclophosphamide monohydrate;
from 20% w/w to 99% w/w dehydrated alcohol; and
from 2% w/w to 5% w/w water.

10. The stable cyclophosphamide solution of claim 3, comprising:
from 2% w/w to 50% w/w cyclophosphamide monohydrate;
from 20% w/w to 99% w/w dehydrated alcohol;
from 2% w/w to 5% w/w water; and
from 0.01% w/w to 5% w/w monothioglycerol.

11. The stable cyclophosphamide solution of claim 1, comprising:
about 22.2% w/w cyclophosphamide monohydrate;
about 74.81% w/w dehydrated alcohol; and
about 3% w/w water.

12. The stable cyclophosphamide solution of claim 1, further comprising a pharmaceutically acceptable carrier.

13. The stable cyclophosphamide solution of claim 12, wherein the pharmaceutically acceptable carrier is selected from the group consisting of sugar; starch; cellulose and its derivatives; powdered tragacanth; malt; gelatin; talc; excipients; oils; glycols; esters; agar; buffering agents; alginic acid; pyrogen-free water; isotonic saline; Ringer's solution; ethyl alcohol; phosphate buffer solutions; lubricants; coloring agents; releasing agents; coating agents; sweetening agents; flavoring agents; perfuming agents; preservatives; antioxidants; and a combination of two or more thereof.

14. The stable cyclophosphamide solution of claim 1, further comprising a stabilizer.

15. A stable cyclophosphamide solution for parenteral administration comprising cyclophosphamide in the form of pharmaceutically acceptable hydrate and/or anhydrous forms in ethanol at about 5% w/w to 50% w/w, water at a level between 2.0% and 5.0% and one or more antioxidants at a level of monothioglycerol at about 0.01% to 5% w/w and butylated hydroxytoluene at about 0.001% to 1% w/w.
